(19) United States
(12) Patent Application Publication
(10) Pub. No.: US 2026/0047849 A1
(43) Pub. Date: Feb. 19, 2026

(54) ACCESS DEVICE FOR USE WITH AN OCCLUSIVE MEMBER DELIVERY SYSTEM

(71) Applicant: BOSTON SCIENTIFIC SCIMED, INC., Maple Grove, MN (US)

(72) Inventors: Sara Reda Eskander, Plymouth, MN (US); David John Onushko, Maple Grove, MN (US); Nicholas Lee Tassoni, Andover, MN (US)

(73) Assignee: BOSTON SCIENTIFIC SCIMED, INC., Maple Grove, MN (US)

(21) Appl. No.: 19/297,305

(22) Filed: Aug. 12, 2025

Related U.S. Application Data
(60) Provisional application No. 63/682,413, filed on Aug. 13, 2024.

(51) Int. Cl. A61B 17/12; A61M 25/01; A61M 25/06

(52) U.S. Cl. CPC A61B 17/12172; A61B 17/12031; A61B 17/12122; A61M 25/0147; A61M 25/0662; A61B 2017/1205; A61M 2025/0681; A61M 2205/0222

(57) ABSTRACT

Access devices for use with an occlusive implant delivery system are disclosed. An example access device may include an elongate sheath having a distal end region, a distal end, and defining a lumen therethrough. A steering member may be coupled to the elongate sheath and disposed adjacent to the distal end of the elongate sheath. A steering wire may be coupled to the steering member and may extend proximally therefrom. The steering wire may have a distal region where the steering wire is engaged with an inner wall surface of the distal end region of the elongate sheath and a friction-reduced region proximal of the distal region.

### CROSS REFERENCE TO RELATED APPLICATIONS

The present application claims the benefit of U.S. Provisional Patent Application Ser. No. 63/682,413, filed Aug. 13, 2024, the disclosure of which is incorporated herein by reference.

### TECHNICAL FIELD

The present disclosure pertains to medical devices, and methods for manufacturing medical devices. More particularly, the present disclosure pertains to access devices for use with an occlusive member delivery system.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An access device for use with an occlusive implant delivery system is disclosed. The access device comprises: an elongate sheath having a distal end region, a distal end, and defining a lumen therethrough; a steering member coupled to the elongate sheath and disposed adjacent to the distal end of the elongate sheath; a steering wire coupled to the steering member and extending proximally therefrom; and wherein a steering wire has a distal region where the steering wire is engaged with an inner wall surface of the distal end region of the elongate sheath and a friction-reduced region proximal of the distal region.

Alternatively or additionally to any of the embodiments above, the distal region of the steering wire is secured to the inner wall surface of the distal end region of the elongate sheath.

Alternatively or additionally to any of the embodiments above, the distal region of the steering wire is bonded to the inner wall surface of the distal end region of the elongate sheath.

Alternatively or additionally to any of the embodiments above, the distal region of the steering wire is adhesively bonded to the inner wall surface of the distal end region of the elongate sheath.

Alternatively or additionally to any of the embodiments above, the distal region of the steering wire is thermally bonded to the inner wall surface of the distal end region of the elongate sheath.

Alternatively or additionally to any of the embodiments above, the distal region of the steering wire includes a surface texture that increases a frictional engagement between the distal region of the steering wire and the inner wall surface of the distal end region of the elongate sheath.

Alternatively or additionally to any of the embodiments above, the elongate sheath includes a deflection point (e.g., a pre-defined deflection point), and wherein the deflection point is disposed proximally of the steering member.

Alternatively or additionally to any of the embodiments above, the deflection point (e.g., the pre-defined deflection point) is disposed adjacent to a distal end of the friction-reduced region of the steering wire.

Alternatively or additionally to any of the embodiments above, a friction-reducing tubular member is disposed over the friction-reduced region of the steering wire.

Alternatively or additionally to any of the embodiments above, a stop member is coupled to the distal region of the steering wire, wherein the stop member is configured to reduce translation of the distal region of the steering wire relative to the friction-reducing tubular member.

Alternatively or additionally to any of the embodiments above, the stop member includes a lug.

Alternatively or additionally to any of the embodiments above, the stop member includes a coil.

A steerable access device with a pre-determined deflection region is disclosed. The access device comprises: an elongate sheath having a distal end region and a distal end; a steering member coupled to the elongate sheath and disposed adjacent to the distal end of the elongate sheath; a steering wire coupled to the steering member and extending proximally therefrom; wherein a steering wire has a distal region and a friction-reduced region; a friction-reducing tubular member disposed over the friction-reduced region of the steering wire; and wherein the pre-determined deflection region is defined adjacent to a distal end of the friction-reducing tubular member.

Alternatively or additionally to any of the embodiments above, the distal region of the steering wire is engaged with an inner wall surface of the distal end region of the elongate sheath.

Alternatively or additionally to any of the embodiments above, the distal region of the steering wire is secured to an inner wall surface of the distal end region of the elongate sheath.

Alternatively or additionally to any of the embodiments above, a stop member is coupled to the distal region of the steering wire, wherein the stop member is configured to reduce translation of the distal region of the steering wire relative to the friction-reducing tubular member.

Alternatively or additionally to any of the embodiments above, the stop member includes a lug.

Alternatively or additionally to any of the embodiments above, the stop member includes a coil.

A method for delivering an occlusive implant is disclosed. The method comprises: advancing an access device within a vascular region and adjacent to a target within a heart of a patient, the access device comprising: an elongate sheath having a distal end region, a distal end, and defining a lumen therethrough, a steering member coupled to the elongate sheath and disposed adjacent to the distal end of the elongate sheath, a steering wire coupled to the steering member and extending proximally therefrom, and wherein a steering wire has a distal region where the steering wire is engaged with an inner wall surface of the distal end region of the elongate sheath and a friction-reduced region proximal of the distal region; and advancing an occlusive device delivery system through the lumen of the elongate sheath.

Alternatively or additionally to any of the embodiments above, advancing an access device within a vascular region and adjacent to a target within a heart of a patient includes actuating the steering wire to deflect the elongate sheath.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example delivery system.

FIG. 2 is a side view of an example delivery system.

FIG. 3 is a side view of a portion of an example access sheath.

FIG. 4 is a cross-sectional view taken through line 4-4 in FIG. 3.

FIG. 5 is a side view of a portion of an example access sheath.

FIG. 6 is a side view of a portion of an example access sheath.

FIG. 7 is a cross-sectional view of a portion of an example access sheath.

FIG. 8 is a cross-sectional view of a portion of an example access sheath.

FIG. 9 is a cross-sectional view of a portion of an example access sheath.

FIG. 10 is a cross-sectional view of a portion of an example access sheath.

FIG. 11 is a cross-sectional view of a portion of an example access sheath.

FIG. 12 is a side view of a portion of an example access sheath.

FIG. 13 is a side view of a portion of an example access sheath.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

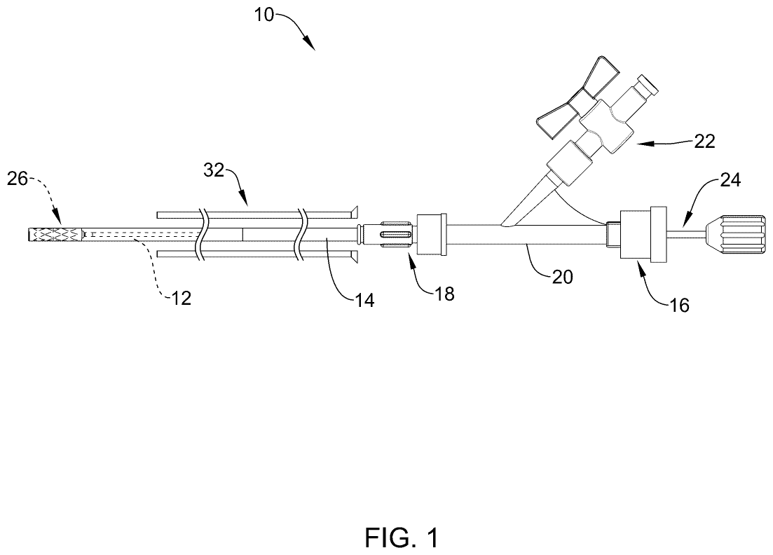

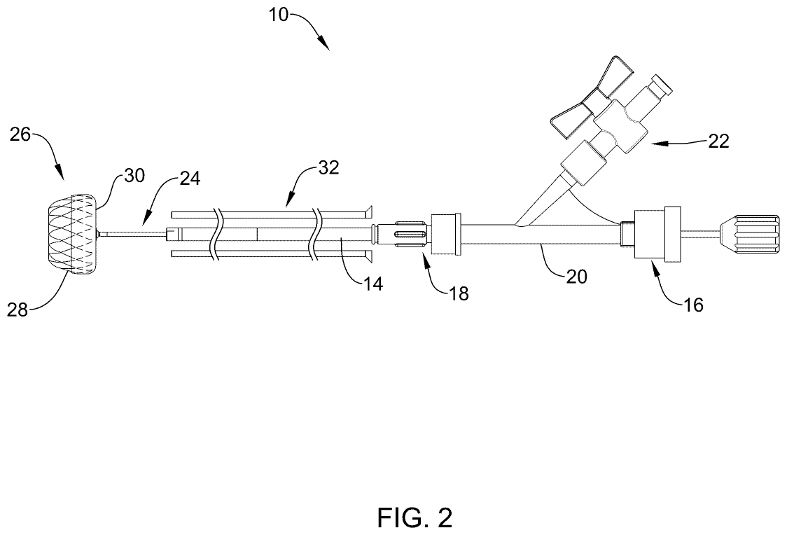

FIGS. 1-2 schematically illustrate selected components and/or arrangements of an occlusive implant system. It should be noted that in any given figure, some features of the occlusive implant system may not be shown, or may be shown schematically, for simplicity. Additional details regarding some of the components of the occlusive implant system may be illustrated in other figures in greater detail. The occlusive implant system may be used to deliver and/or deploy a variety of medical implants (e.g., a cardiovascular implant, an occlusive implant, etc.) to one or more locations within the anatomy, including but not limited to, in some instances, the heart and/or the left atrial appendage. In the interest of clarity, the following discussion refers to an occlusive implant, but other medical implants may be used and/or considered with the occlusive implant system. Example occlusive implants include WATCHMAN FLX™ and WATCHMAN FLX™ Pro from Boston Scientific.

The occlusive implant system may include a delivery system 10 including a delivery sheath 14 having a delivery lumen 12 extending proximally from a distal end of the delivery sheath 14. In one example, the delivery lumen 12 extends from a proximal opening to a distal opening of the delivery sheath 14. The delivery system 10 may include a proximal hub 16. In some instances, the delivery system may include a mid-hub 18. In some instances, the delivery system 10 may include a mid-shaft 20 extending from the proximal hub 16 to the mid-hub 18. In some instances, the delivery sheath 14 may extend distally from the mid-hub 18. Other configurations are also contemplated. In some instances, the delivery system 10 may include a side port 22. In some instances, the side port 22 may be in communication with the mid-shaft 20. Other configurations are also contemplated. In some instances, the delivery system 10 and/or the delivery lumen 12 may include a proximal segment (not shown) extending within and/or through the mid-hub 18, the mid-shaft 20, and the proximal hub 16. In some instances, the proximal segment may be in fluid communication with and/or may be an extension of the delivery lumen 12 of the delivery sheath 14. In some instances, the side port 22 may be in fluid communication with the proximal segment and/or the delivery lumen 12.

The occlusive implant system and/or the delivery system 10 may include a core member or core wire 24 slidably and/or rotatably disposed within the delivery lumen 12 (and the proximal segment, where present). The occlusive implant system may include an occlusive implant 26, which may be configured for implantation within a left atrial appendage, releasably engaged with and/or releasably attached to a distal end of the core wire 24. In at least some embodiments, the occlusive implant 26 may be a left atrial appendage closure device. In some instances, a proximal end of the core wire 24 may extend proximally of a proximal end of the delivery sheath 14 and/or the proximal opening of the delivery lumen 12 for manual manipulation by a clinician or practitioner. In at least some embodiments, the delivery sheath 14 may comprise and/or may be formed from a polymeric material. In some instances, the delivery sheath 14 may comprise and/or may be formed from a plurality of polymeric materials. In some instances, the delivery sheath may comprise and/or may be formed from a combination of metallic and polymeric materials. In some instances, the delivery sheath 14 may include a reinforcing element, such as a mesh, a coil, a braid, etc., formed therein, embedded therein, attached thereto, etc. along at least a portion of a length of the delivery sheath 14. Other configurations are also contemplated. Some suitable, but non-limiting, examples of materials for the occlusive implant system, the core wire 24, and/or the delivery sheath 14, etc., including but not limited to metallic materials, polymeric materials, etc., are discussed below.

The occlusive implant 26 may include an expandable framework 28 (e.g., FIG. 2) configured to shift between a delivery configuration (e.g., FIG. 1), such as when the occlusive implant 26 is disposed within the delivery lumen 12 proximate the distal opening and/or within a distal portion of the delivery lumen 12, and a deployed configuration (e.g., FIG. 2) when the occlusive implant 26 is unconstrained by the delivery sheath 14.

In some instances, the expandable framework 28 may comprise a plurality of interconnected struts. In some instances, the expandable framework 28 may be compliant or semi-compliant and may generally conform to and/or be configured to sealingly engage with the shape and/or geometry of the left atrial appendage in the deployed configuration.

In some instances, a proximal end of the expandable framework 28 may be configured to releasably attach, join, couple, engage, or otherwise connect to the distal end of the core wire 24 (e.g., FIG. 2). In some instances, the proximal end of the expandable framework 28 may include a proximal hub coupled and/or non-releasably attached thereto. In some instances, the proximal hub may be configured to and/or adapted to releasably couple with, join to, mate with, or otherwise engage a distal end of the core wire 24. Other means of releasably coupling and/or engaging the expandable framework 28 to the distal end of the core wire 24 are also contemplated.

In some instances, the occlusive implant 26 may include an occlusive element 30 (e.g., a membrane, a fabric, or a tissue element, etc.) connected to, disposed on, disposed over, disposed about, or covering at least a portion the expandable framework 28. In some instances, the occlusive element 30 may be connected to, disposed on, disposed over, disposed about, or cover at least a portion of an outer (or outwardly facing) surface of the expandable framework 28.

In some instances, the occlusive element 30 may be permeable or impermeable to blood and/or other fluids, such as water. In some instances, the occlusive element 30 may include a polymeric membrane, a metallic or polymeric mesh, a porous or semi-porous filter-like material, or other suitable construction. In some instances, the occlusive element 30 prevents thrombi (e.g., blood clots, etc.) from passing through the occlusive element 30 and out of the left atrial appendage into the blood stream. In some instances, the occlusive element 30 promotes endothelization after implantation, thereby effectively removing the target site (e.g., the left atrial appendage, etc.) from the patient's circulatory system. Some suitable, but non-limiting, examples of materials for the occlusive element 30 are discussed below.

In some instances, the expandable framework 28 and/or the plurality of interconnected struts may be integrally formed and/or cut from a unitary member. In some instances, the expandable framework 28 and/or the plurality of interconnected struts may be integrally formed and/or cut from a unitary tubular member and subsequently formed and/or heat set to a desired shape in the deployed configuration. In some instances, the expandable framework 28 and/or the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the expandable framework 28 include laser cutting, machining, punching, stamping, electro discharge machining (EDM), chemical dissolution, etc. Other means and/or methods are also contemplated.

In use, the delivery sheath 14 may be advanced and/or navigated to the left atrial appendage to deliver the occlusive implant 26 thereto. In one example, the delivery sheath 14 may be advanced and/or navigated to the left atrial appendage using and/or over a guidewire. For example, the delivery sheath 14 may be advanced to the patient's left atrium and the distal end disposed adjacent to the left atrial appendage with the occlusive implant 26 disposed therein in the delivery configuration. In some instances, the delivery sheath 14 may include steering capability. After the distal end of the delivery sheath 14 is disposed adjacent to and/or at the left atrial appendage, the core wire 24 may be advanced distally relative to the delivery sheath 14 to advance the occlusive implant 26 out of the delivery sheath 14, where the occlusive implant 26 may shift to the deployed configuration.

In some instances, the delivery system 10 may further comprise an access sheath 32. For example, the access sheath 32 may be advanced and/or navigated toward the left atrial appendage. In some instances, the access sheath 32 may include steering capability. During use, the delivery sheath 14 may be advanced within the access sheath 32 with the occlusive implant 26 disposed therein in the delivery configuration. After the distal end of the delivery sheath 14 is disposed adjacent to and/or at the distal end of the access sheath 32, the core wire 24 may be advanced distally relative to the delivery sheath 14 and/or the access sheath 32 to advance the occlusive implant 26 out of the delivery sheath 14 and the access sheath 32, where the occlusive implant 26 may shift to the deployed configuration. Disclosed herein are access sheaths that may, for example, be used in conjunction with an occlusive device and/or a delivery system for delivering an occlusive device/implant.

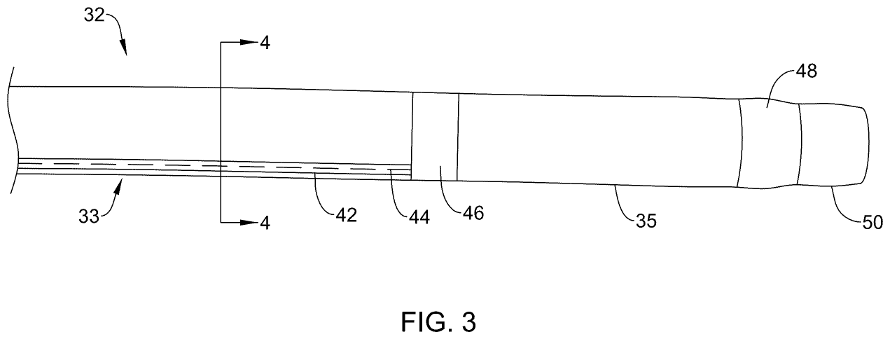

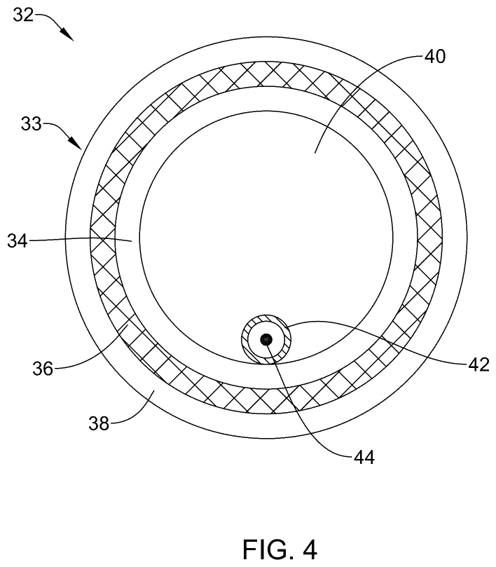

An example access sheath 32 is schematically depicted in FIGS. 3-4. The access sheath 32 includes a sheath or sheath body 33 having a distal end region 35. A lumen 40 may be defined through the sheath 33. A marker member 48 (e.g., a radiopaque marker formed from or including a radiopaque material) may be coupled to the sheath 33, for example adjacent to a distal end region or tip 50 of the sheath 33. The form of the access sheath 32 may vary. For example, as shown in FIG. 4 the sheath 33 may be formed from a plurality of layers such as an inner layer or liner 34, a reinforcing member or layer 36, and an outer layer 38. The inner layer 34 may include a lubricious material such as polytetrafluoroethylene. Other materials including those disclosed herein may be utilized. The reinforcing layer 36 may include a braid or coil. The outer layer may include a polyether block amide. Other materials including those disclosed herein may be utilized.

A low friction tube 42 may be disposed within the lumen 40. The low friction tube 42 may resemble a small/miniature catheter shaft and may be disposed along the inner wall of the sheath 33. In some instances, the low friction tube 42 may be formed from a single layer of lubricious material or a number of layers. For example, the low friction tube 42 may include an inner lubricious layer (e.g., formed from polytetrafluoroethylene or the like), a reinforcing braid, and an outer layer formed from polyether block amide. Other materials including those disclosed herein may be utilized.

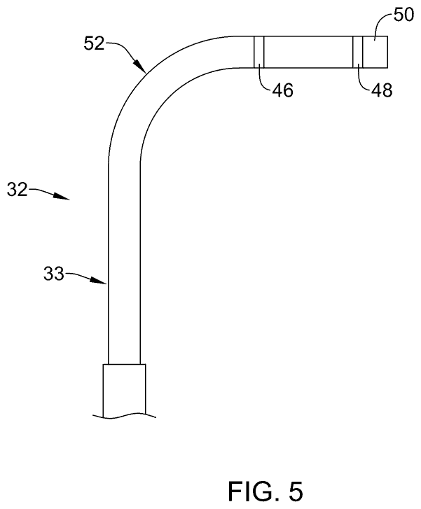

A steering wire 44 may be disposed within the low friction tube 42. As the name suggests, the steering wire 44 may be used to steer, bend, and/or otherwise deflect the sheath 33. As shown in FIG. 3, the steering wire 44 (e.g., a distal end of the steering wire 44) may be coupled to a steering member 46. A proximal end (not shown) of the steering wire 44 may be accessible to a clinician so that actuating/pulling the steering wire 44, deflects the sheath 33 as depicted in FIG. 5. Because the steering member 46 is positioned proximally of the distal tip 50, a deflection point or region 52 (shown in FIG. 5) may be defined in the sheath 33. In some instances, the deflection point 52 may be positioned about 15-50 mm proximally of the distal tip 50. The deflection point 52 being positioned proximally of the distal tip 50 may be desirable for a number of reasons. For example, a proximally positioned deflection point 52 may allow the access sheath 32 to be navigated toward a left atrial appendage. In at least some instances, the deflection point 52 may be understood to be a pre-defined and/or pre-determined deflection point. For the purposes of this disclosure, a pre-defined and/or pre-determined deflection point is a deflection point that is defined at a particular axial region of the sheath 33, for example through manufacturing, so that the sheath 33 has a tendency to preferentially bend/deflect at the deflection point 52 when the steering wire 44 is actuated as opposed to other locations along the length of the sheath 33. In other words, actuation of the steering wire 44 tends to deflect the sheath 33 at the (e.g., pre-defined and/or pre-determined) deflection point 52. In at least some instances, it may be desirable for the sheath 33 to bend/deflect at a position that is proximal of the distal tip 50. Thus, by having a pre-defined deflection point, the deflection point 52 can be controlled so that deflection preferentially occurs along the desired location of the sheath 33 (e.g., proximal of the distal tip 50). Some examples for how the sheath 33 and/or other sheaths disclosed herein are structurally arranged and/or manufactured in order to include a pre-defined and/or pre-determined deflection point are disclosed herein.

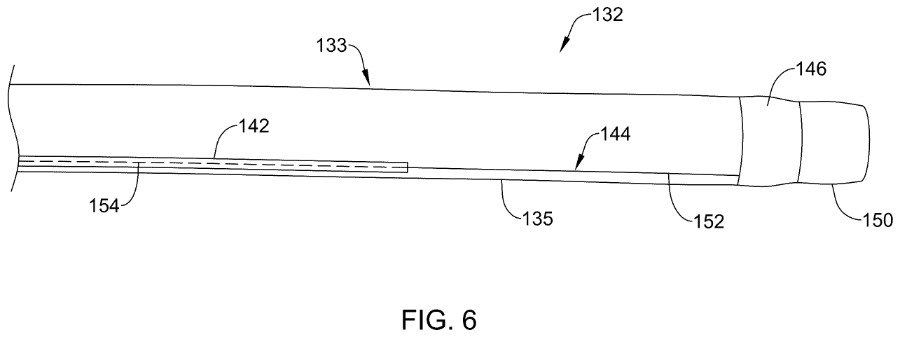

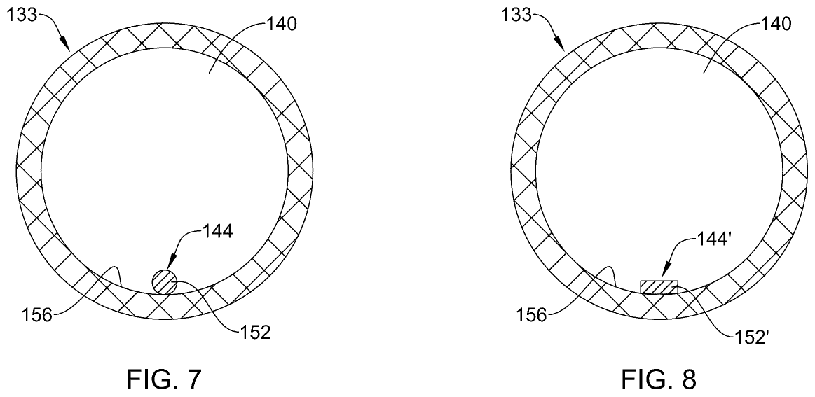

FIG. 6 illustrates another access sheath 132 that may be similar in form and function to other access sheaths disclosed herein. The access sheath 132 may include a sheath or sheath body 133 having a distal end region 135. A lumen 140 (see, for example, FIG. 7) may be defined in the sheath 133. A low friction tube 142 may be disposed along an inner wall of the sheath 133. A steering wire 144 may extend through the low friction tube 142. The steering wire 144 may be coupled to a steering member 146. In this example, the steering member 146 may be disposed adjacent to a distal end region or tip 150 of the sheath 133. This may be desirable for a number of reasons. For example, positioning the steering member 146 adjacent to the distal tip 150 may aid in simplifying manufacturing of the access sheath 132 and/or reduce costs/labor that may be associated with manufacturing the access sheath 132.

As indicated herein, it may be desirable for the access sheath 132 to have or include a deflection point/region (e.g., a pre-defined and/or pre-determined deflection point/region similar to the deflection point 52) that is positioned proximally of the distal tip 150. One way that it may be possible to have a deflection point/region that is positioned proximally of the distal tip 150 while having the steering member 146 positioned adjacent to the distal tip 150 is to engage, secure, and/or otherwise limit axial translation of a portion of the steering wire 144. For example, the steering wire 144 may include a distal region 152 where the steering wire 144, for example, is engaged with an inner wall surface of the distal end region 135 of the sheath 133. The steering wire 144 may also include a friction-reduced region 154 proximal of the distal region 152. The friction-reduced region 154 may extend through the low friction tube 142. It can be appreciated that the friction-reduced region 154 of the steering wire 144 may be understood to be the portion of the steering wire 144 within the low friction tube 142. For the purpose of this disclosure, the friction-reduced region 154 may be understood to have reduced friction by virtue of a coating disposed along the wire. In some of these and in other instances, the friction-reduced region 154 may be reduced in friction simply because the friction-reduced region 154 is disposed within the low friction tube 142. In other words, the friction-reduced region 154 has a lowered/reduced friction and/or defines a lower/reduced friction region of the steering wire 144 due to the low friction tube 142 and not necessarily because of any friction-reducing structure/element disposed along the steering wire 144. The distal region 152 of the steering wire 144 may be understood to be the portion of the steering wire 144 extending distally from the distal end of the low friction tube 142. In practice, the friction-reduced region 154 of the steering wire 144 is intended to be translatable relative to the sheath 133 so that the sheath 133 can be steered, bent, and/or otherwise deflected. The distal region 152, as indicated herein, is intended to be engaged with the sheath 133, secured to the sheath 133, and/or otherwise designed with increased friction so as to limit axial translation thereof relative to the sheath 133.

Disposing the distal region 152 directly against the inner wall and/or bonding (e.g., thermal bonding, adhesive bonding, reflowed with the sheath 133, and/or the like, etc.) the distal region 152 to the inner wall may help to limit the axial translation of the distal region 152 relative to the sheath 133. For example, FIG. 7 depicts the distal region 152 of the steering wire 144 bonded to (e.g., thermally bonded, adhesively bonded, reflowed with the sheath 133, and/or the like, etc.), disposed against, and/or in contact with the inner wall surface 156 of the sheath 133. The engagement of the distal region 152 with the inner wall surface 156 helps to increase friction between the distal region 152 of the steering wire 144 and the inner wall surface 156 of the sheath 133 and/or otherwise limit the axial translation of the distal region 152 relative to the sheath 133. By doing so, the deflection point/region (e.g., similar to the deflection point 52) can be defined in the access sheath 132 at a position proximal of the distal tip 150. Actuation of the steering wire 144, thus, causes the access sheath 132 to steer, bend, and/or deflect. Because the axial translation of the distal region 152 of the steering wire 144 is reduced relative to the sheath 133, the deflection point/region (e.g., similar to the deflection point 52) can be defined in the access sheath 132 at a position proximal of the distal tip 150.

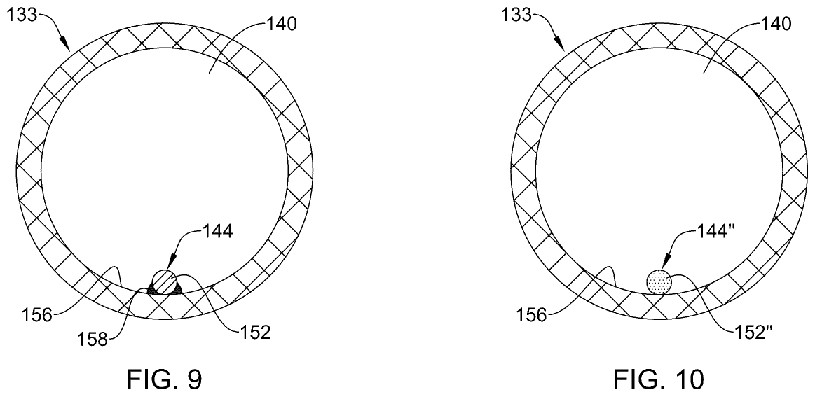

A number of additional ways to increase the friction between the distal region 152 of the steering wire 144 and the inner wall surface 156 of the sheath 133 are contemplated. For example, FIG. 8 illustrates a distal region 152′ of a steering wire 144′ that is ribbon shaped. The ribbon shape of the distal region 152′ may help to increase friction and/or limit translation of the distal region 152′ relative to the sheath 133. In some of these and in other instances, the distal region 152′ may be bonded to (e.g., thermally bonded, adhesively bonded, reflowed with the sheath 133, and/or the like, etc.) the sheath 133. In some of these and in other instances, the distal region 152′ may be sized and/or shaped so as to resist entering the low friction tube 142. For example, the distal region 152′ may have a larger diameter than the inner diameter of the low friction tube 142 and/or the distal region 152′ may have a different shape (e.g., a ribbon shape) that substantially prevents the distal region 152′ from being retracted into the low friction tube 142. Similarly, FIG. 9 depicts the use of an adhesive 158 to secure the distal region 152 of the steering wire 144 to the inner wall surface 156 of the sheath 133. The adhesive 158, which is shown schematically, may represent the use of an adhesive substance to form and adhesive bond or may be understood to represent the distal region 152 being thermally bonded with or reflowed with the sheath 133.

Another example is depicted in FIG. 10, where a distal region 152″ of a steering wire 144″ includes a surface treatment or texture that helps to increase the friction between the distal region 152″ of the steering wire 144″ and the inner wall surface 156 of the sheath 133. In this example, the surface treatment or texture is represented by speckled line shown along the steering wire 144″.

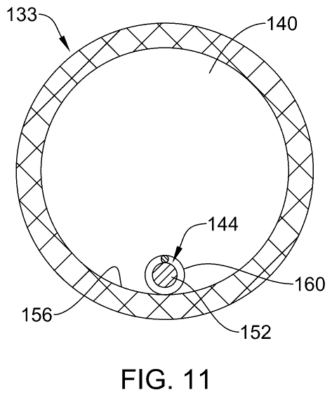

FIG. 11 illustrates another example where a coil member 160 may be disposed along a portion of the distal region 152 of the steering wire 144. In some instances, the coil member 160 may have a discrete length that extends along only a portion of the distal region 152 of the steering wire 144 (e.g., which may include the coil member 160 being coupled to and/or attached to the distal region 152 of the steering wire 144). In other instances, the coil member 160 may have a longer length and may extend, for example substantially the entire length of the distal region 152. The coil member 160 may help to increase the friction between the distal region 152 of the steering wire 144 and the inner wall surface 156 of the sheath 133. In some of these and in other instances, the coil member 160 may also aid in bonding the distal region 152 of the steering wire 144 to the inner wall surface 156 of the sheath 133, for example to limit axial translation of the distal region 152. In at least some instances, the coil member 160 may be sized in order to substantially prevent the distal region 152 of the steering wire 144 from being pulled into the low friction tube 142. For example, the coil member 160 may be bonded to the distal region 152 of the steering wire 144 and may function as a physical barrier that substantially prevents the distal region 152 of the steering wire 144 from being retracted into the low friction tube 142.

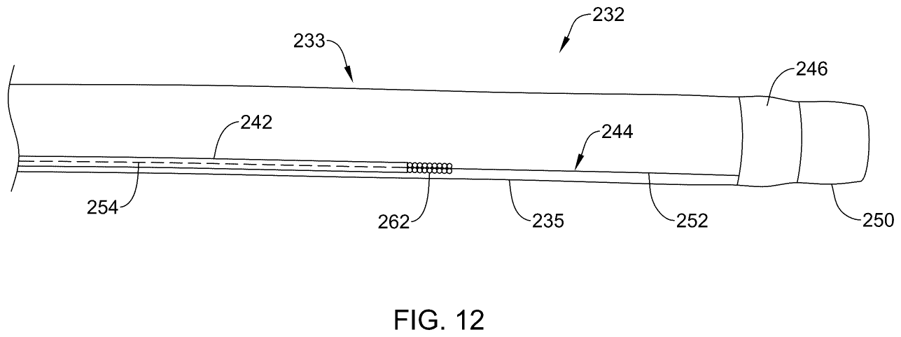

FIG. 12 illustrates another access sheath 232 that may be similar in form and function to other access sheaths disclosed herein. The access sheath 232 may include a sheath or sheath body 233 having a distal end region 235. A low friction tube 242 may be disposed along an inner wall of the sheath 233. A steering wire 244 may extend through the low friction tube 242. The steering wire 244 may be coupled to a steering member 246. In this example, the steering member 246 may be disposed adjacent to a distal end region or tip 250 of the sheath 233. This may be desirable for a number of reasons. For example, positioning the steering member 246 adjacent to the distal tip 250 may aid in simplifying manufacturing of the access sheath 232 and/or reduce costs/labor that may be associated with manufacturing the access sheath 232.

As indicated herein, it may be desirable for the access sheath 232 to have or include a deflection point/region (e.g., a pre-defined and/or pre-determined deflection point/region similar to the deflection point 52) that is positioned proximally of the distal tip 250. For example, the steering wire 244 may include a distal region 252 where the steering wire 244, for example, is engaged with an inner wall surface of the distal end region 235 of the sheath 233. The steering wire 244 may also include a friction-reduced region 254 proximal of the distal region 252. The friction-reduced region 254 may extend through the low friction tube 242.

In the example depicted in FIG. 12, a coil member 262 may be coupled to the steering wire 244, for example the distal region 252 of the steering wire 244. In some instances, the coil member 262 may have a discrete length that extends along only a portion of the distal region 252 of the steering wire 244. In other instances, the coil member 262 may have a longer length and may extend, for example substantially the entire length of the distal region 252 (e.g., from the distal end of the low friction tube 242 to the steering member 246. The coil member 262 may function, for example, by being a physical barrier or interfering structure that prevents the distal region 252 of the steering wire 244 from being retracted into the low friction tube 242. Thus, the coil member 262 may help to limit axial translation of the distal region 252 of steering wire 244 relative to the sheath 233. This, much like in other instances disclosed herein, can help to define a deflection point/region (e.g., similar to the deflection point 52) in the access sheath 232 at a position proximal of the distal tip 250.

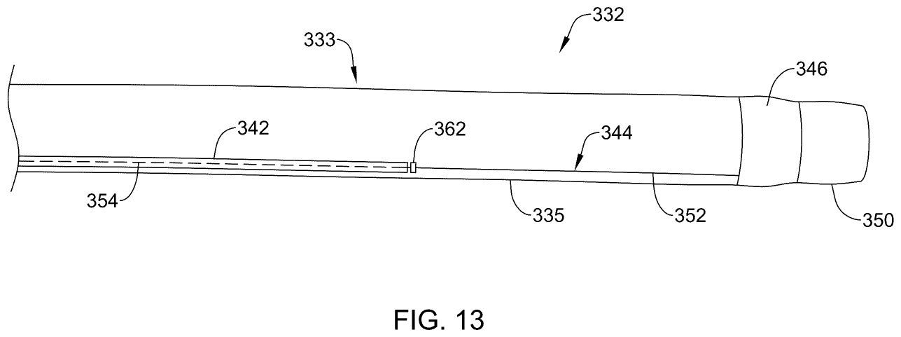

FIG. 13 illustrates another access sheath 332 that may be similar in form and function to other access sheaths disclosed herein. The access sheath 332 may include a sheath or sheath body 333 having a distal end region 335. A low friction tube 342 may be disposed along an inner wall of the sheath 333. A steering wire 344 may extend through the low friction tube 342. The steering wire 344 may be coupled to a steering member 346. In this example, the steering member 346 may be disposed adjacent to a distal end region or tip 350 of the sheath 333. This may be desirable for a number of reasons. For example, positioning the steering member 346 adjacent to the distal tip 350 may aid in simplifying manufacturing of the access sheath 332 and/or reduce costs/labor that may be associated with manufacturing the access sheath 332.

As indicated herein, it may be desirable for the access sheath 332 to have or include a deflection point/region (e.g., a pre-defined and/or pre-determined deflection point/region similar to the deflection point 52) that is positioned proximally of the distal tip 350. For example, the steering wire 344 may include a distal region 352 where the steering wire 344, for example, is engaged with an inner wall surface of the distal end region 335 of the sheath 333. The steering wire 344 may also include a friction-reduced region 354 proximal of the distal region 352. The friction-reduced region 354 may extend through the low friction tube 342.

In the example depicted in FIG. 13, a stop member 362 (e.g., lug, coil, stopper, etc.) may be coupled to the steering wire 344, for example the distal region 352 of the steering wire 344. The stop member 362 may function, for example, by being a physical barrier or interfering structure that prevents the distal region 352 of the steering wire 344 from being retracted into the low friction tube 342. Thus, the stop member 362 may help to limit axial translation of the distal region 352 of steering wire 344 relative to the sheath 333. This, much like in other instances disclosed herein, can help to define a deflection point/region (e.g., similar to the deflection point 52) in the access sheath 332 at a position proximal of the distal tip 350.

The materials that can be used for the various components of the systems disclosed herein may include those commonly associated with medical devices. For simplicity purposes, the following discussion refers to the sheath 33 and other components of the access sheath 132. However, this is not intended to limit the devices and methods described herein, as the discussion may be applied to other similar sheaths or access sheaths disclosed herein.

The sheath 33 and/or other components of the access sheath 132 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some instances, the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the access sheath 132 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the access sheath 132 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the access sheath 132 to achieve the same result.

In some instances, a degree of Magnetic Resonance Imaging (MRI) compatibility is imparted into the access sheath 132. For example, the access sheath 132, or portions thereof, may be made of a material that does not substantially distort the image and create substantial artifacts (e.g., gaps in the image). Certain ferromagnetic materials, for example, may not be suitable because they may create artifacts in an MRI image. The access sheath 132, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An access device for use with an occlusive implant delivery system, the access device comprising:
an elongate sheath having a distal end region, a distal end, and defining a lumen therethrough;
a steering member coupled to the elongate sheath and disposed adjacent to the distal end of the elongate sheath;
a steering wire coupled to the steering member and extending proximally therefrom; and
wherein a steering wire has a distal region where the steering wire is engaged with an inner wall surface of the distal end region of the elongate sheath and a friction-reduced region proximal of the distal region.

2. The access device of claim 1, wherein the distal region of the steering wire is secured to the inner wall surface of the distal end region of the elongate sheath.

3. The access device of claim 1, wherein the distal region of the steering wire is bonded to the inner wall surface of the distal end region of the elongate sheath.

4. The access device of claim 3, wherein the distal region of the steering wire is adhesively bonded to the inner wall surface of the distal end region of the elongate sheath.

5. The access device of claim 3, wherein the distal region of the steering wire is thermally bonded to the inner wall surface of the distal end region of the elongate sheath.

6. The access device of claim 1, wherein the distal region of the steering wire includes a surface texture that increases a frictional engagement between the distal region of the steering wire and the inner wall surface of the distal end region of the elongate sheath.

7. The access device of claim 1, wherein the elongate sheath includes a deflection point, and wherein the deflection point is disposed proximally of the steering member.

8. The access device of claim 7, wherein the deflection point is disposed adjacent to a distal end of the friction-reduced region of the steering wire.

9. The access device of claim 1, wherein a friction-reducing tubular member is disposed over the friction-reduced region of the steering wire.

10. The access device of claim 9, wherein a stop member is coupled to the distal region of the steering wire, wherein the stop member is configured to reduce translation of the distal region of the steering wire relative to the friction-reducing tubular member.

11. The access device of claim 10, wherein the stop member includes a lug.

12. The access device of claim 10, wherein the stop member includes a coil.

13. A steerable access device with a pre-determined deflection region, the access device comprising:
an elongate sheath having a distal end region and a distal end;
a steering member coupled to the elongate sheath and disposed adjacent to the distal end of the elongate sheath;
a steering wire coupled to the steering member and extending proximally therefrom;
wherein a steering wire has a distal region and a friction-reduced region;
a friction-reducing tubular member disposed over the friction-reduced region of the steering wire; and
wherein the pre-determined deflection region is defined adjacent to a distal end of the friction-reducing tubular member.

14. The access device of claim 13, wherein the distal region of the steering wire is engaged with an inner wall surface of the distal end region of the elongate sheath.

15. The access device of claim 13, wherein the distal region of the steering wire is secured to an inner wall surface of the distal end region of the elongate sheath.

16. The access device of claim 13, wherein a stop member is coupled to the distal region of the steering wire, wherein the stop member is configured to reduce translation of the distal region of the steering wire relative to the friction-reducing tubular member.

17. The access device of claim 16, wherein the stop member includes a lug.

18. The access device of claim 16, wherein the stop member includes a coil.

19. A method for delivering an occlusive implant, the method comprising:
advancing an access device within a vascular region and adjacent to a target within a heart of a patient, the access device comprising:
an elongate sheath having a distal end region, a distal end, and defining a lumen therethrough,
a steering member coupled to the elongate sheath and disposed adjacent to the distal end of the elongate sheath,
a steering wire coupled to the steering member and extending proximally therefrom, and
wherein a steering wire has a distal region where the steering wire is engaged with an inner wall surface of the distal end region of the elongate sheath and a friction-reduced region proximal of the distal region; and
advancing an occlusive device delivery system through the lumen of the elongate sheath.

20. The method of claim 19, wherein advancing an access device within a vascular region and adjacent to a target within a heart of a patient includes actuating the steering wire to deflect the elongate sheath.
